(19) United States
(12) Patent Application Publication
(10) Pub. No.: US 2008/0118727 A1
(43) Pub. Date: May 22, 2008

(54) PROCESS FOR PRODUCING ELASTIC AND/OR WATER DEGRADABLE WEBS FROM COMPOSITE FILAMENTS

(76) Inventor: Jens Ole Brochner ANDERSEN, Skanderborg (DK)

Correspondence Address: WINSTON & STRAWN LLP;PATENT DEPARTMENT, 1700 K STREET, N.W., WASHINGTON, DC, 20006, US

(21) Appl. No.: 11/858,537

(22) Filed: Sep. 20, 2007

Related U.S. Application Data
(63) Continuation of application No. PCT/EP2006/061095, filed on Mar. 28, 2006.

(30) Foreign Application Priority Data
Mar. 31, 2005 (EP) EP 05075753.3

(51) Int. Cl. A61F 13/15; B29C 67/00

(52) U.S. Cl. 428/219; 428/221; 264/125

(57) ABSTRACT

A process of manufacturing a non-woven web from virtually endless composite filaments. The filaments used in the process are arranged in a sheath-core arrangement in which the sheath component comprise a thermoplastic polymer and the core component is selected from the group of an elastomer, a water-soluble polymer or a biodegradable polymer. The sheath component constitutes at least 20 percent by weight of the filament and that the core component constitutes at least 10 percent by weight of the filament. The process according to the invention provides a simple and inexpensive process for manufacturing water soluble or elastic non-woven webs of any width, using virtually endless filaments.

### CROSS-REFERENCE TO RELATED APPLICATIONS

This application is a continuation of International application PCT/EP2006/061095 filed Mar. 28, 2006, the entire content of which is expressly incorporated herein by reference thereto.

### FIELD OF INVENTION

The present invention relates to composite filaments and to a process for producing elastic, water-soluble or water degradable webs from the filaments. The invention further relates to the webs obtainable by the process and the use of the non-woven webs.

### BACKGROUND OF THE INVENTION

Non-woven webs are used in the manufacture of a variety of products such as bandaging materials, garments, diapers, incontinence products, support clothing, and personal hygiene products. These articles are normally designed to absorb and contain bodily fluids and at the same time provide a physical barrier to such fluids. In order to allow more freedom of body movement, the articles can advantageously be elastic.

Products of the kind named above are conventionally disposed as normal household waste, and thereafter either placed in landfills or combusted. Either way, the waste constitutes a potential environmental hazard, and the demand to reduce the amount of everyday waste is growing.

Manufactures of the article are presently trying to solve these problems by making the articles flushable. It should in this respect be noted, that there are no common definition of the term flushable. The term is used at random when a product fits down the toilet, not taking into consideration what happens to the product after it enters the sewage system. While there are several types of flushable articles on the market for toilet cleaning, kids care and personal hygiene, these articles are not fully degradable and are therefore a threat to municipal septic and sewer systems nationally.

Non-woven webs are conventionally produced by a variety of methods, but only the well known “spunbond” process is capable of utilizing long fiber filaments. In the “spunbond” process, filaments of one or more molten polymers are extruded from a large number of orifices formed in a spinnerette plate. The filaments are immediately thereafter stretched or drawn, and are then randomly deposited upon a collection surface to form a non-woven web. The stretching or attenuation can be mechanically through the use of draw rolls, or, as is more widely practiced, pneumatically by passing the filaments through a pneumatic attenuator.

Manufacturers of spunbonded non-woven fabrics have long sought to improve the manufacturing process to achieve higher productivity and better quality and uniformity of the spunbonded non-woven fabric. Maintaining the quality and uniformity of the fabric becomes a particular concern at higher production speeds and when producing fabrics of low basis weight.

While spunbond materials with desirable combinations of physical properties, especially combinations of softness, strength and durability, have been produced, significant problems have also been encountered. One main problem is attributed to the fact that the width of the non-woven webs manufactured with the spundbonding process is limited by the width of the spinnerette plate, as this plate has to be arranged across the whole width of the production line. Manufacturing broad non-woven webs by such process therefore requires large unrenunerative or uneconomical production plants. It has therefore in practice, been necessary to reach a compromise where a plant is operated with relatively small width of web, resulting in that the capacity of the plant is far from being fully utilized.

Another problem with the spundbond process relates to the manufacture of e.g. elastic non-woven webs. Elastic fibers have a characteristic “sticky” nature, as these fibers normally comprise elastomers. Spunbonding, which employ air drawing, can be particularly effected. For example, turbulence in the air can bring filaments into contact and these “sticky” filaments can then adhere to one another. This stickiness proves to be especially troublesome during winding of the webs into rolls. The layers of web adhere to one another, a phenomenon known as “blocking”.

While it can be possible to decrease the effect of the stickiness of the elastic filaments, this involves further process steps, and therefore introduces a significant complication into the process for producing an elastic non-woven web. Such complications can result in a significant addition to the cost of the resulting fabric. Separately, attempts have been made to influence the properties of fabrics by modifying the content of the fibers, e.g. by combining polymers in bi- and multicomponent fibers.

### DESCRIPTION OF THE PRIOR ART

One example of a bi-component elastic fiber is known from U.S. Pat. No. 5,352,518, and use of such filaments in the spundbonding process reduces some of the drawbacks, but the limited production width of the web using the spundbonding process still adds additional costs to the final product.

The known bi-component filaments only have a very thin sheath surrounding the core, and these known filaments have therefore not been able to produce non-woven webs having the desirable combinations of physical properties, especially combinations of softness, strength and durability, as most of the properties of the final web are provided by the core component. Furthermore, these known filaments also faced problems such as breakage or elastic failure of the strand during extrusion and/or drawing. Broken strands can clog the flow of filaments and/or mesh with other filaments, resulting in the undesired formation of a mat of tangled filaments in the web.

While the art has sought to address the foregoing problems, it is clear that the results have, at best, been mixed. Thus, improvements in this area are necessary and desired.

### SUMMARY OF THE INVENTION

The present invention now provides a simple and inexpensive process for manufacturing non-woven webs of any width, using virtually endless filaments. These webs are water soluble and elastic and are produced at low cost.

The invention also a novel elastic filament, one that is both a water-soluble and biodegradable filament. These filaments can be provided in a non-woven web which gives an excellent feeling to wearers and which is fully degradable in water.

The new and unique way in which the present invention fulfills the above mentioned aspects is that the composite filaments are arranged in a sheath-core arrangement, wherein the sheath component comprises at least one thermoplastic polymer and the core component comprises at least one elastomer, at least one water-soluble polymer, or at least one biodegradable polymer or combinations thereof, and that the sheath component constitutes at least 20 percent by weight of the filaments while the core component constitutes at least 10 percent by weight of the total weight of the filaments.

It has been surprisingly found that when the sheath component is present in an amount of at least 20 percent by weight, based on the total weight of the filament, the filaments have the advantage, compared to conventional composite filaments, that they will not break during their preparation, i.e., during the extrusion and/or drawings step of the manufacture. These filaments will therefore never clog the flow of filaments and/or mesh with other filaments, and the problem with tangled filaments is therefore eliminated.

Furthermore, the relatively high amount of the sheath component in respect of the total weight of the filament also influence the properties of the final product, as both the sheath- and core component in a much higher degree than hitherto known contributes to the properties of the web.

Depending on the desired application of the filament, it is advantageous in one embodiment according to the invention, that the contents of the sheath component is at least 30 percent by weight of the total weight of the filament, preferably at least 40 percent by weight of the total weight of the filament, more preferably at least 50 percent by weight of the total weight of the filament, alternatively at least 60 percent by weight of the total weight of the filament, preferably at least 70 percent by weight of the total weight of the filament, alternatively at least 80 percent by weight of the total weight of the filament or at least 90 percent by weight of the total weight of the filament.

The amount of the sheath component of the total filament is according to the invention selected in order to both prevent that the filaments clog the flow of filaments and/or mesh with other filaments during the manufacture of the filaments and also that the final web obtains the desired properties. Filaments having the above-mentioned composition are capable of providing a non-woven web with desirable combinations of physical properties, especially combinations of softness, strength and durability.

The filaments according to the invention can be used in a process for manufacturing a non-woven web, the process comprises the following steps, defibrating the filaments, transporting the defibrated filaments to at least one forming head and forming a non-woven web on an endless forming wire.

During the initial defibrating step the virtually endless filaments will be divided into smaller segments and/or fibers, enabling these fibers to be used in e.g. a conventional airlaying process. Thereby is not only obtained the advantage that elastic webs and/or water soluble webs can be formed in a more simple and more economical process than hitherto known, but also, that virtually endless filaments can be used in these process.

It should in this respect be mentioned, that conventional airlaying processes in some instances include a defibration step, however this conventional step is included in the process in order to unwind and open fluff pulp, and not, as in the present invention, to defibrate long filaments. The difference can especially be found as no rolled up fiber lumps, collectively known as nits, are formed during the defibration of the filaments, which normally possess an extreme problem during the conventional defibration of fluff pulp.

In the process according to the invention the filaments are defibrated before they enter the forming heads. Furthermore, the process according to the invention provides the advantages, that the width of the web can be much broader, as the spinneret used to manufacture the filaments have no effect on the dimensions of the final web, as in the conventional spunbonding process. The spinneret used to prepare the filaments before they are being defibrated, can therefore have a lesser dimension, ensuring that the spinneret occupies lesser space in the plant.

Alternatively, the spinneret can be separate from the production plant, as the filaments do not have to be produced simultaneously with the web. Furthermore, filaments of different weights and/or physical and/or chemical properties can in an advantageously embodiment be defibrated in the process according to the invention either simultaneously or at different stages of the process. Thereby, the process according to the invention is not only very flexible, as webs will several layers with different properties or weights easily can be produced with the process according to the invention, the process according to the invention is also a more simple and economical process than hitherto known.

As an example can be mentioned, that the filaments e.g. can be produced with weights from 0.3 dtex to 30 dtex, i.e. 10,000 meters of the filaments weights from 0.3 to 30 g, respectively, and that webs manufactured with these filaments provide webs with characteristics and qualities not previously known from corresponding webs.

The process according to the invention can further include opening and feeding short cut staple fiber and dose superabsorbents or other powders to one or more forming heads. These materials can be suspended in air within a forming system and deposited on a moving forming screen or rotating perforated cylinder.

As the sheath component of the filaments comprises a thermoplastic polymer this polymer will be activated during a subsequent thermal bonding step. During the step, the web can e.g. pass through a through-air oven, which activates thermoplastic sheath component of the defibrated filaments, binding the web components together. As the sheath component is present in an amount of at least 20 percent by weight of the total weight of the filament, i.e. the amount of sheath component is much higher than in conventional bicomponent fibers, thermal bonding step will ensured, that the defibrated filaments are bonded much more efficiently together than hitherto known, and that both the properties of the sheath- and core component can be utilized optimally.

After activation the product can be calendered to the correct thickness and cooled before it is winded into jumbo rolls. Thermally bonding and calendaring can advantageously be applied in one step, by a heated calendar. To have an economic process the sheath component should preferably have a lower melting temperature than the core component, and in a preferred embodiment thermoplastic polymer is a polyamide with a very low melting point, e.g. a polyester or a polyolefin. The specific melting point will depend on the selected polymer and the degree of e.g. branching but the polyamide will preferably be selected to have a melting point in the range of about of about 60° C. to 220° C. The polyester will advantageously have a melting point in the range of about 180° C. to 220° C. and the polyolefin a melting point in the range of about 60° C. to about 115° C.

During thermal bonding step the sheath polymer will melt and be concentrated in the junctions between the fibers, thereby, at least partly, uncovering the core component. The properties of both the sheath- and the core component can then be utilized optimally, while at the same time obtaining a strong web. The person skilled in the art would understand, that the process according to the present inventions could utilize either bi- or mulicomponent filaments. Furthermore, the core component does not have to be a single unit but can be made up of several independent elements, giving the filament an inlands-in-the sea construction. The different element can in a preferred embodiment be composed of the same or different polymer/elastomers. Furthermore, the different elements can be either uniformly or randomly distributed in the sheath component. Similar, the sheath component can be composed of several different layers, or can be a mixture of different thermoplastic polymers.

The nature of the final web are determined by the nature of the filaments, thus when the core component is an elastomer the final web will be an elastic web and when the core component is a water-soluble polymer and/or a biodegradable polymer the web will e.g. be capable of dissolving in water. The resultant web will preferably have a final weight of the web in the range between 20 and 500 g/m², depending on the final use, and can comprise a number of different layers.

According to one embodiment of the present invention the core component is an elastomer. By elastomer is meant an amorphous, cross-linked high polymer which will stretch rapidly under tension, reaching high elongations (500 to 1000%) with low damping. It has high tensile strength and high modulus when fully stretched. On the release of stress, it will retract rapidly, exhibiting the phenomenon of snap or rebound, to recover its original dimensions. Elastomers are unlike thermoplastics in that they can be repeatedly softened and hardened by heating and cooling without substantial change in properties.

When the core component is an relatively inexpensive elastomer, e.g. a polyolefin such as polypropylene or a styrenic elastomer, the resultant webs can advantageously be used as disposable articles such as diapers, training pants or incontinence garments. The elastomer will provide the articles with a close, comfortable fit about the wearer and contain body exudates while maintaining skin health.

In other more durable products, such as waist elastics, leg elastics, elasticized liners, and elasticized outer covers i.e. elastic products with multi-use applications, the elastic condensation polymers such as polyurethane and copolyester, can advantageously be applied. These elastic components are employed to help produce and maintain the fit of the articles about the body contours of the wearer thereby leading to improved containment and comfort.

The elastic web of an embodiment of the present invention can be combined with one or more webs to provide a soft texture that may be more useful or appealing in some applications. Such webs can be fibrous in nature, examples being nonwoven and woven materials. One embodiment of the invention includes a composite material that comprises the elastic web described previously and an additional web. The composite material may be prepared by laminating the webs together, coextrusion, or any other suitable method for making the composite material.

Embodiments of the present invention provide elastic materials that contain apertures and are breathable when stretched, and in particular, breathable when stretched by a tensile force acting in the direction of the force that the material would experience in end use conditions (e.g., in a diaper side tab that would normally experience the hoop stress of the diaper waist band when gripping the wearer's waist). Another example of stress in the direction of the force that the material would experience in end use conditions includes the stress that would be experienced by a bandage that is wrapped in around a body part, or that is stretched and then adhered.

In another embodiment according to the present invention the core component is a water-soluble polymer and/or a biodegradable polymer, ensuring that the web will disintegrate when it comes into contact with water. The core component can be of any material that is adequately soluble and that will give appropriate properties to the final product. Preferably it has low oxygen permeability when dry. It can be, for instance, a polyethylene oxide (PEO) or a polyvinyl alcohol (PVOH).

PVOH are generally made by hydrolysis from polyvinyl acetate and the degree of hydrolysis affects solubility. Thus the degree of hydrolysis can be selected depending on the application of the final product.

Fully hydrolyzed PVOHs (e.g., hydrolyzed to an extent of at least about 98%) tend to be readily soluble only in warm or hot water. Thus if the final product are to be used as e.g. water soluble toilet paper it is preferred to use grades of polyvinyl alcohol which are not quite so fully hydrolyzed, as the less hydrolyzed grades tend to dissolve more readily in cold water and water with room temperature, e.g. 10° C. to 25° C. Therefore partially hydrolyzed PVOH is preferably used, preferably having a degree of hydrolysis from polyvinyl acetate of 70 to 95%, most preferably 73 to 93%, when the product are to be applied in normal daily necessities.

PVOH used alone as a base polymer for the formation of a water-soluble web in the conventional techniques, suffers from several disadvantages. Due to PVOHs high melting point and poor thermal stability, it is very difficult to thermally process. An extruder, rather than merely a melt tank, is required to process the PVOH into a web. Additionally, once the web is formed, it has poor heat seal properties such that it would need to be heat sealed at temperatures that adversely affect the integrity of the substrate. The problems are solved by the present inventions, as the PVOH is sheathed with thermoplastic polymer, ensuring that the PVOH easily can be processed into a thermally stabilized web.

The products comprising the water-soluble and/or degradable polymer produced according the present invention has a modified rate of water solubility i.e. they can both withstand to be exposed to the extremely varied strength requirements in the wet and dry states and at the same time dissolve in water after a specific time. The water-soluble core component will namely be in direct contact with the water, as thermal sheath polymer has melted during thermal bonding step and concentrate in the junctions between the fibers, whereby the core component is at least partly uncovered. The features of the core component can then be utilized optimally while at the same time obtaining a strong web.

For ensuring, that the webs according to the invention retain their strength at least for a specific period of time when exposed to aqueous liquids or moisture-containing food, the invention can adventurously comprise means for delaying the disintegrating when the article comes into contact with water. This can for instance be relevant in the case of household paper (kitchen towels). On the other hand, toilet paper must dissolve in water, some time after use, in order to prevent the sewage systems from clogging up. At the same time, wet toilet paper must not immediately loose its strength properties during use for apparent reasons.

Correspondingly, the prior art makes a distinction between dry strength and wet strength properties, the latter being divided in further categories such as initial wet strength, temporary wet strength and permanent wet strength depending on the point of time of measuring the wet strength after re-wetting a dry tissue paper.

In one embodiment the means for delaying the disintegration in water is a thin surface-coating, which is applied to the final article via conventional techniques. This ensures that the article is both capable of keeping the strength properties during use and at the same time that the article is capable of disintegrating in water. An example of such surface is a latex coating, but other coatings providing the same or similar properties can equally well be used. Coatings of this type are well known to the person skilled in the art.

As an alternative to a surface coating, the product could e.g. be premoistened with a stabilizing solution and/or wet-strength additives, which is not capable of dissolving the core component or sheath-polymer.

If the web comprises PVOH, the web can advantageously be premoistened with a stabilizing solution having a low salt concentration, as the salt will stabilize the bindings in the web. When the web is placed in contact with water having a lower salt concentration, the salt will be washed out, and the article will disintegrate.

Alternatively the article can be stabilized with calcium ions, which also stabilize the bindings in the web. When the article is immersed in water with less calcium or an excess of sodium ions, the solubility of the article increases.

If the polymer is PEO and/or PVOH the agent could preferably be saline with a relatively low salt concentration, of e.g. 1 M NaCl.

In preparing the premoistened article according to the invention, any of various suitable methods may be used. For example, the web may be saturated with the stabilizing solution and then encapsulated or otherwise sealed in an airtight liquid impermeable package. The premoistened article of the invention is ideally suited to be carried by a person in a packet or purse and, because it is premoistened, it is available immediately for use for wiping in a one-step cleaning operation.

Wet strength is an important characteristic of non-woven products. Using wet strength additives can increase wet strength of such products. The most widely used wet streak additives for the non-woven industry are melamine-formaldehyde and urea-formaldehyde, however the person skilled in the art would understand that other commercially availably wet-strength additives also could be used with similar effect. Dry and wet strength properties can e.g. be determined using the Hercules method for Paper Strength Testing.

In one embodiment of the invention a liquid disinfectant and/or deodorizer is added to the premoistened stabilizing solution, whereby the article functions to effectively cleanse, disinfect and deodorize.

The filaments according to the invention can preferable by used to produce articles designed to e.g. absorb and contain bodily fluids and/or provide a physical barrier to such fluids e.g. diapers, personal hygiene products or sanitary napkins.

The non-woven webs according to the invention can further be used in the manufacture of bandaging materials, garments, and support clothing.

### BRIEF DESCRIPTION OF THE FIGURES

The invention will be explained in greater detail below where further advantageous properties and example embodiments are described with reference to the examples and drawing, wherein:

FIGS. 1A-B, schematically illustrates the structure of two different embodiments of the filament according to the invention,

FIG. 2 is an electron-microscopy picture of an elastic web according to the invention.

FIG. 3 is an electron-microscopy picture of a biodegradable web according to the invention.

### DETAILED DESCRIPTION OF THE PREFERRED EMBODIMENTS

Below, the invention is described on the assumption that the core component and sheath component is circular, however the invention is not limited to this specific structure. Thus, the component and/or sheath component can have other structures, such as hexagonal or triangular or islands-in-the-sea structures with similar, or in some cases better, technical advantages, depending on the resultant web.

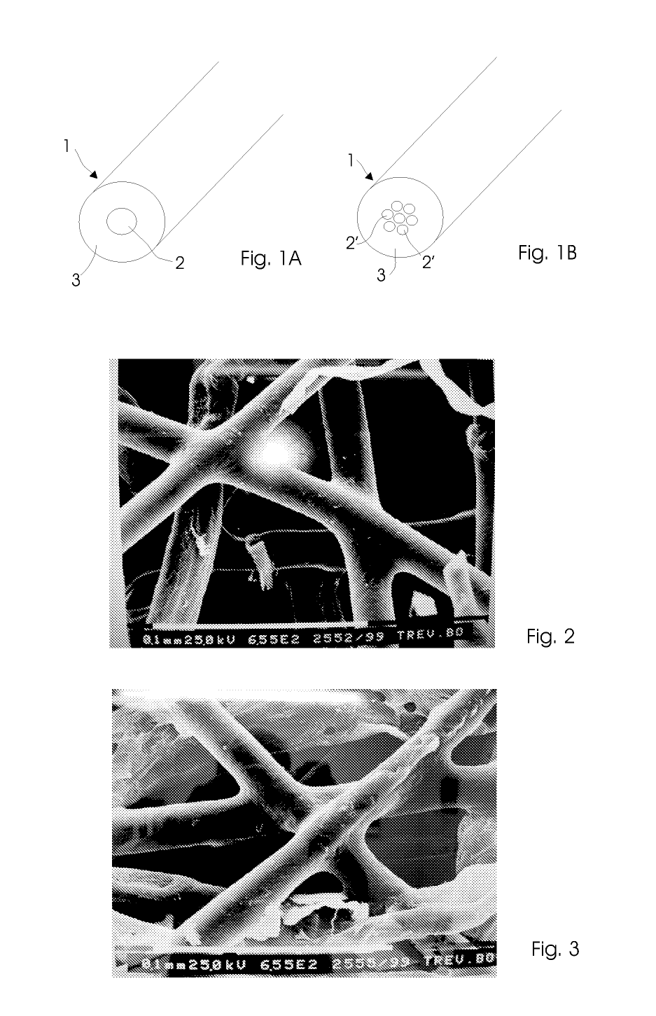

FIG. 1A is a schematic view of a filament 1 according to the invention. The filament 1 is designed with a core component 2 and a circumferential sheath component 3.

The sheath component 3 comprises a thermoplastic polymer and the core component 2 can be an elastomer, a water-soluble polymer and/or a biodegradable polymer, depending on the desired features of the final product.

In FIG. 1B is the core component 2 divided into a number of core-elements 4, 5 uniformly distributed in the center of the sheath component 3. In the present case is part of the elements an elastomer 4, and the rest of the elements an water degradable polymer 5. The sheath component 3 is spread between the elements 4,5. When the sheath polymer melts during thermal bonding step the different core-elements 4,5 will be exposed, and the resultant web will be both elastic and water-degradable.

FIGS. 2 and 3 are respectively electron-microscopy pictures of elastic and a biodegradable webs according to the invention. As illustrated by the arrows in the figures, it is evident, that the sheath component which has been melted during thermal bonding step, has flow towards the junctions between the fibers where it has concentrated, thereby uncovering the core component 2, at least partly. The properties of the core component will then be able to be utilized optimally, while at the same time obtaining a strong web.

### EXAMPLES

The following examples further illustrate the preferred embodiments of the invention.

### Example 1

### Manufacture of the Filament

Fiber material having the general configuration of a sheath-core arrangement is prepared from molten polymers of the respective sheet-core polymers.

The molten polymers are formed in a batch process were they are forced through an extrusion head forming a spaghetti type product, which is cooled down and passed through a chip cutter where it is cut into so called chips.

In order to manufacture the bicomponent fiber material the different chips are fed onto two separate extruders, one for the sheet component and one for the core component. Electrically heated zones around the cylinder in the extruder and high pressures caused by the action of the screw melt the chips and a fairly thick liquid results. The heating system keeps it in a molten state while it is fed at a controlled rate via spin or metering pumps into spin packs.

The molten polymers are forced through the spinnerette holes in the spin packs at a defined speed. To obtain the correct fiber thickness a constant pulling force is exerted by a roller arrangement, which draw the fibers down the spinning shafts.

The fibers formed by the spinnerette are still liquid and can adventurously be rapidly cooled down in order to solidify. For these purposes quench air is blown through the fiber bundle.

### Example 2

### Air Laying of the Web

The resulting filaments are fiberized in a defibration unit, and the resulting fibers are thereafter supplied to a forming head in the air laying plant by a fiber transport fan. The plant can be a multiple forming head systems. When each head is fed with its own unique blend of raw materials, it is possible to produce multilayer products, where each layer is engineered for a specific function in the product, for instance acquisition-distribution layer, absorption layer, barrier layer etc.

### Example 3

### Elastic and/or Water Soluble Non-Woven Webs

A bicomponent polyethylene filament (PEO-1), comprising 65-percent by weight polyolefin as a sheath component and 35-percent by weight polyethylene oxide as the core polymer was prepared as described in example 1. The total weight of the filament was 15 dtex.

A bicomponent polypropylene fiber material (PP-1), comprising 65-percent by weight polyolefin as a sheath component and 35-percent by weight polypropylene as the core polymer was prepared as described in example 1. The total weight of the filament was 30 dtex.

PEO-1 and PP-1 was used to produced a number of different webs, either alone, in combination or in blends with other material and/fibers, such as SAP, cellulose fibers.

All webs are manufactured according to the process described in Examples 1 and 2, resulting in webs with the following characteristics:

### Web 1

Wipes (120 g/m²) comprising between 15 and 25 percent by weight PEO-1, 0 to 15 percent by weight liquid binder and 60 to 85 percent by weight cellulose fiber were prepared. These wipes all showed a significant low wet strength and were completely disintegrated in tap water after only few minutes. These webs can therefore be considered completely flushable.

### Web 2

Wipes (220 g/m²) prepared from a homogenous web comprising 15 to 50 percent by weight PEO-1 and 50 to 85 percent by weight cellulose fiber. These wipes were not only soft but were also capable of being disintegrated in tap water.

### Web 3

However, a homogenous web (140 g/m²) with 100 percent by weight PEO-1, was not only disintegrated in water after few minutes, it was also very elastic.

### Web 4

Homogenous web (80 g/m²) with 50 percent by weight PEO-1 and 50 percent by weight elastic PP-1. This web was both elastic and capable of disintegrating in water.

### Web 5

Web (360 g/m²) comprising 35 to 65 percent by weight cellulose fiber, 35 to 65 percent by weight absorbent layer (SAP) and 3 to 15 percent by weight PEO-1. Also when the web comprised SAP was the web capable of being disintegrated in water.

### Web 7

| | |
|---|---|
| Top layer | 100 percent by weight synthetic PEO-1 |
| (20 g/m²) | |
| Middle layer | 35 to 65 percent by weight cellulose fiber, 35 to 65 to |
| (350 g/m²) | percent by weight absorbent layer and 3 to 15 percent |
| | by weight PEO-1. |
| Bottom layer | 100 percent by weight very fine dtex PEO-1. |
| (30 g/m²) | |

This web has a low wet strength ensuring that it was completely disintegrated in tap water after very few minutes.

### Web 8

| | |
|---|---|
| Top layer | 100 percent by weight synthetic PEO-1 |
| (40 g/m²) | |
| Middle layer | 35 to 65 percent by weight cellulose fiber, 35 to 65 |
| (220 g/m²) | percent by weight absorbent layer and 3 to 15 percent |
| | by weight PEO-1 |
| Bottom layer | 50 percent by weight very fine dtex PEO-1 and 50 |
| (40 g/m²) | percent by weight PP-1. |

This web showed a low wet strength and were completely disintegrated in tap water after only very few minutes. The web further exhibited excellent elastic properties.

While particular embodiments of the present invention have been illustrated and described, it would be obvious to those skilled in the art that various other changes and modifications can be made without departing from the scope of the invention. It is therefore intended to cover in the appended claims all such changes and modifications that are within the scope of this invention.

What is claimed is:

1. A non-woven web comprising at least one composite filament arranged in a sheath-core arrangement, wherein the sheath component comprises at least one thermoplastic polymer and the core component comprises at least one water-soluble polymer, with the sheath component constituting at least 20 percent by weight of the filaments and the core component constituting at least 10 percent by weight of the filaments, wherein the at least one water soluble polymer is selected in order to ensure that the non-woven web will disintegrate when the non-woven web comes into contact with water having a temperature at or below room temperature.

2. The non-woven web according to claim 1, wherein the least one water soluble polymer is a polyethylene oxide or a polyvinyl alcohol.

3. The non-woven web according to claim 2, wherein the polyvinyl alcohol is partly hydrolyzed, having a degree of hydrolysis from polyvinyl acetate of 70 to 95%.

4. The non-woven web according to claim 2, wherein the polyvinyl alcohol is partly hydrolyzed, having a degree of hydrolysis from polyvinyl acetate of 73 to 93%.

5. The non-woven web according to claim 1 wherein the sheath component has a lower melting temperature than the core component.

6. The non-woven web according to claim 1 wherein the at least one thermoplastic polymer is a polyamide.

7. The non-woven web according to claim 1 premoistened with one of a stabilizing solution, a wet-strength additive, or both.

8. The non-woven web according to claim 1 wherein the non-woven web has a weight of between 20 and 500 g/m².

9. A process of manufacturing the non-woven web according to claim 1, which comprises:
defibrating the filaments,
transporting the defibrated filaments to at least one forming head, and
distributing the defibrated filaments on an endless forming wire, placed beneath the at least one forming head.

10. The process according to claim 9, wherein the defibrated filaments has a fiber length between about 0.5 and about 12 mm.

11. The process according to claim 9, wherein the defibrated filaments has a fiber length between about 1 mm and about 10 mm.

12. The process according to claim 9, wherein the defibrated filaments has a fiber length between about 3 and about 9 mm.

13. The process according to claim 9 wherein the process further comprises a thermal bonding step.

14. The process according to claim 9, wherein the temperature of thermal bonding step is higher than the melting temperature of the sheath component and lower than the melting temperature of the core component.

15. A flushable article comprising the non-woven web according to claim 1.

16. The flushable article according to claim 15, in the form of a diaper, incontinence product, cleaning pad or personal hygiene product.
